(19) United States
(12) Patent Application Publication
(10) Pub. No.: US 2020/0275831 A1
(43) Pub. Date: Sep. 3, 2020

(54) LIGHTED SPECULA, RELATED KITS, AND METHODS FOR USING THE SAME

(71) Applicant: Progressive Innovations, LLC, Carmel, IN (US)

(72) Inventor: Howard Levitin, Carmel, IN (US)

(21) Appl. No.: 16/646,430

(22) PCT Filed: Sep. 11, 2018
(86) PCT No.: PCT/US18/50348

§ 371 Date: Mar. 11, 2020

Related U.S. Application Data
(60) Provisional application No. 62/556,745, filed on Sep. 11, 2017.

(51) Int. Cl. A61B 1/32; A61B 1/00; A61B 1/06; A61B 1/233

(52) U.S. Cl. CPC A61B 1/32; A61B 1/00032; A61B 1/233; A61B 1/0684; A61B 1/00039

(57) ABSTRACT

Lighted speculum devices comprising a dilation means and an integral light source are provided. The light source may be directional, such that light is emitted through the distal end of the dilation means, and/or diffuse, such that light emits both through the distal end of the dilation means and the walls thereof. The lighted speculum is also coupled with a power source and a switching mechanism, all of which is configured to be operable using one hand. Methods for dilating and visualizing a body cavity are also provided, as well as kits to facilitate the commercial use of the presently disclosed lighted speculum.

### PRIORITY

The present application is related to and claims the priority benefit of U.S. Provisional Patent Application Ser. No. 62/556,745 to Levitin, filed on Sep. 11, 2017. The content of the aforementioned application is hereby expressly incorporated by reference in its entirety into this disclosure.

### BACKGROUND

Specula are medical tools for investigating body orifices or cavities and come in a variety of shapes and sizes based on their intended purpose. A speculum allows entry into a body cavity such that a practitioner can obtain direct vision of an area of interest and, if desired, introduce instruments for further interventions (e.g., such as a biopsy). Conventional specula generally consist of two hinged blades that are configured to move between a “closed” configuration to facilitate entry into a body orifice and an “opened” configuration to dilate the body orifice when the speculum is in its final position. In certain designs, one of the blades may be stationary relative to the speculum handle and one pivoting, such that only the pivoting blade moves away from and towards the stationary blade to open and close the speculum, respectively. Alternatively, such as in conventional nasal specula, both blades may be moveable relative to each other.

The size and lengths of the blades are typically configured to correlate with the targeted orifice and/or the particular patient. For example, vaginal specula comprise longer blades for accessing the cervix, whereas nasal specula comprise shorter blades to accommodate narrow nasal passageways. Conventional specula are typically formed of stainless steel or other types of metal (which can be sterilized and reused) or, the case of vaginal specula in particular, may be formed of plastic.

There are several drawbacks to existing speculum designs. One of the most significant drawbacks is the inability for a practitioner to fully visualize an area of interest within the body cavity. Body cavities are inherently dark, which often impairs visualization of the targeted body area. To overcome this, practitioners often wear a headlamp or utilize external light sources to illuminate the body cavity and targeted site. Headlamps and external light sources are not ideal, however, as they are cumbersome and an additional device for the practitioner to manage while performing often sensitive procedures.

Certain specula systems have attempted to provide light by inserting a light source into the speculum such that it sends light through the speculum to illuminate the viewing area. For example, one conventional lighted specula system includes a separate light stick designed for manual insertion into the interior space between the blades when the speculum is in use. This is problematic because it leaves little room within the dilated orifice for any additional tools that may be required for a procedure and the light stick itself can obstruct the practitioner's visualization of the targeted area. The practitioner is also required to manipulate and manage both the speculum and light stick during a sensitive and/or urgent procedure, adding undesirable complexity thereto. Furthermore, the light stick is typically powered by a power source connected via a cord (e.g., a corded plug for coupling with a wall outlet). As the cord is an additional element that must be managed by the practitioner during the procedure and also must be cleaned between each use to avoid cross contamination, this is not ideal. While some conventional lighted specula do have a light coupled directly with the specula itself such that a practitioner need not manipulate a separate lighting device, such conventional specula are bulky and still attached to an external power source via a cord and present the drawbacks related thereto.

### SUMMARY

The devices, kits, and methods of the present disclosure relate to a novel lighted speculum that is inexpensive to manufacture, easy to use, and significantly improves exam area visualization within a body orifice. Certain exemplary embodiments provided herein are also disposable, which negates the risk of contamination due to improper sterilization between procedures.

At least one exemplary embodiment of the present disclosure comprises a lighted speculum device comprising a dilation component configured to move between a closed configuration and an open configuration, at least one handle coupled with a proximal end of the dilation component, a light source (configured to emit light upon activation) coupled with the dilation component, a power source in electrical communication with the light source, and a switching mechanism. The handle of the lighted speculum device may comprise an actuator configured to cause the dilation component to move between the open and closed configurations. The power source of the lighted speculum device may be coupled directly to one or more of the dilation component, the at least one handle, or the light source. Further, the switching mechanism may be configured to allow or disrupt electricity flow between the light source and the power source. In at least one exemplary embodiment, the switching mechanism is operable to activate the light source upon a single touch by a user.

In at least one embodiment, the light source is coupled with the proximal end of the dilation component and the power source comprises one or more batteries. There, for example, each battery may be coupled directly with an inner side of the at least one handle. In at least one embodiment, the light source comprises at least one bulb coupled at or near a proximal end of the dilation component. The light source may be positioned within an interior of the dilation component or otherwise, provided the light emitted by the light source can reach a visualization area defined by the dilation component and/or can transverse a wall (or perforations therein) of the dilation component. In at least one embodiment, the light source is affixed to the dilation component.

The dilation component may comprise a clear or opaque, disposable plastic material. Additionally, or alternatively, the light source may comprise a lighted cable extending along a length of the dilation component. In yet another embodiment, a portion of at least one wall extending between a proximal end and a distal end of the dilation component may comprise perforations and/or a transparent or translucent material configured to allow light to traverse therethrough.

Methods for dilating and visualizing an orifice are also provided. In at least one exemplary embodiment, a method for dilating and visualizing an orifice comprises the steps of: inserting a dilation means of a lighted speculum device into an orifice of a patient, the dilation means positioned in a closed configuration; dilating the orifice of the patient and enlarging a viewing area defined by a distal end of the dilation means by moving the dilation means to an open configuration; activating a light source of the lighted speculum to emit light at least through the distal end of the dilation means and into the orifice; and visualizing a targeted site within the orifice through the viewing area. In at least one exemplary embodiment, a power source coupled with the light source is positioned within or on the lighted speculum.

The device used in the aforementioned method may comprise at least one wall extending between a proximal end of the dilation means and the distal end of the dilation means. Where at least a portion of such wall may comprise a material that allows light to pass therethrough, in at least one exemplary embodiment, the step of activating the light source may further comprise illuminating a portion of the dilated orifice other than the viewing area by emitting diffuse light through the portion of the at least one wall. The light source may be affixed to the dilation means or positioned as otherwise described herein. In at least one exemplary embodiment, the light source comprises a lighted cable or wire that extends along a length of the dilation means.

In at least one embodiment of the method, the step of activating the light source comprises operating a switching mechanism configured to allow or disrupt electrical flow between the light source and the power source.

Where the orifice comprises a nasal cavity, the targeted site may be a bleeding artery, for example, where the method is employed to treat a nose bleed. Where the targeted site is a bleeding artery (irrespective of whether or not the orifice is a nasal cavity), the method may further comprise the step of inserting a cauterization tool through an interior defined by the dilation means and the viewing area, and cauterizing the bleeding artery.

Dilation kits are also provided in the present disclosure. In at least one embodiment, the dilation kits hereof comprise a lighted speculum comprising a dilation component and a light source. There, the dilation component may comprise a proximal end, a distal end that defines a viewing area at least when the dilation component is in an open configuration, and at least one wall that defines an interior space, extends between a proximal end and a distal end, and comprises at least a portion that allows light to pass therethrough. The light source, likewise, may be coupled with the dilation component such that light emitted therefrom illuminates the viewing area and passes through the portion of the at least one wall of the dilation component.

In addition to the foregoing, the dilation kits hereof may further comprise one or more cauterization sticks. There, the distal end of each cauterization stick is configured to be insertable within the interior space and through the viewing area of the dilation component of the lighted speculum. Additionally or alternatively, the dilation kit may comprise a nasogastric tube. In addition to or in lieu of any of the foregoing implements, the dilation kit may comprise one or more of a biopsy tool, a water irrigation device, forceps, and a suction catheter. Still further, where the lighted speculum further comprises at least one battery in electrical communication with the light source and the light source comprises at least one bulb, the kit may further comprise one or more replacement batteries, one or more replacement bulbs, or both.

### BRIEF DESCRIPTION OF THE DRAWINGS

The present invention will be better understood and features, aspects and advantages other than those set forth above will become apparent in light of the following detailed description thereof. Such detailed description makes reference to the following drawings, wherein:

FIG. 1 shows a perspective view of the back of at least one embodiment of a lighted nasal speculum of the present disclosure;

FIG. 2A shows a perspective view of at least one embodiment of the lighted nasal speculum of FIG. 1;

FIG. 2B shows a side view of at least one embodiment of the lighted nasal speculum of FIG. 1;

FIG. 3A shows a front view of at least one embodiment of the lighted nasal speculum of FIG. 1 with the dilation means in an open configuration;

FIG. 3B shows a top view of at least one embodiment of the lighted nasal speculum of the present disclosure with the dilation means in an open configuration;

FIG. 4A shows a perspective view of a lighted speculum according to at least one embodiment of the present disclosure composed of, at least in part, a transparent material;

FIG. 4B shows a perspective view of a lighted speculum according to at least one embodiment of the present disclosure wherein at least the top portion of the dilation means is comprised of a translucent material;

FIG. 5 shows a practitioner dilating a nasal cavity of a patient using a lighted speculum according to at least one embodiment of the present disclosure; and

FIG. 6 shows a flow chart representative of a method of using a lighted speculum according to at least one embodiment of the present disclosure.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the embodiments above and the claims below. Reference should therefore be made to the embodiments above and claims below for interpreting the scope of the invention.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, many modifications and other embodiments of the technology described herein will come to mind to one of skill in the art to which the present disclosure pertains having the benefit of the teachings presented in the present descriptions and associated figures. Therefore, it is understood that this disclosure covers any such alternatives, modifications, and equivalents as may be included within the spirit and scope of this application as defined by the specification and appended claims. While this technology may be illustrated and described in one or more preferred embodiments, devices, kits, and methods hereof may comprise many different configurations, forms, materials, and accessories.

Wherever feasible and convenient, like reference numerals are used in the figures and the description to refer to the same or like parts or steps. The drawings are in a simplified form and not to precise scale. It is understood that the disclosure is presented in this manner merely for explanatory purposes and the principles and embodiments described herein may be applied to devices and/or system components that have dimensions/configurations other than as specifically described herein. It is expressly contemplated that the size and shapes of the composition and system components of the present disclosure may be tailored in furtherance of the desired application thereof.

The devices, kits, and methods of the present disclosure relate to a novel lighted speculum that is inexpensive to manufacture, easy to use, and significantly improves exam area visualization within a body orifice. Certain exemplary embodiments provided herein are also disposable, which negates the risk of contamination due to improper sterilization between procedures.

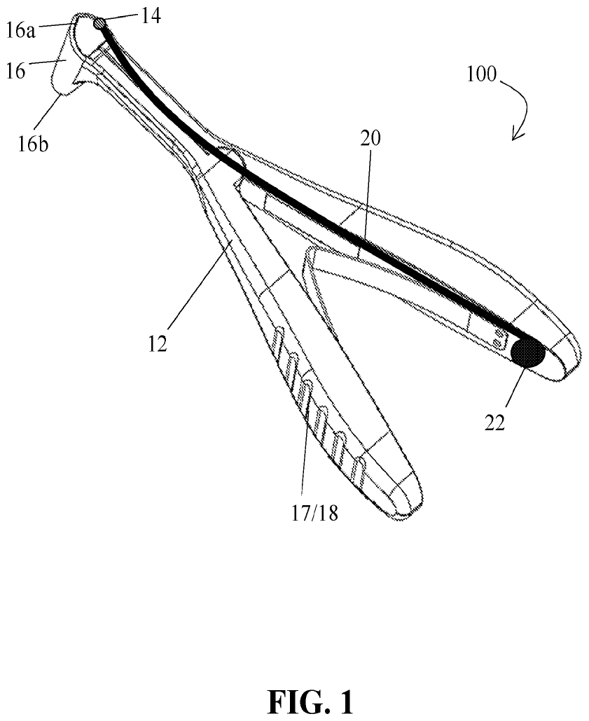

Now referring to FIGS. 1-4B, exemplary embodiments of novel devices 100, 300, 400 of the present disclosure are shown. Devices 100, 300, 400 each generally comprises a speculum 12 with one or more light sources 14 and a power source 22 coupled therewith.

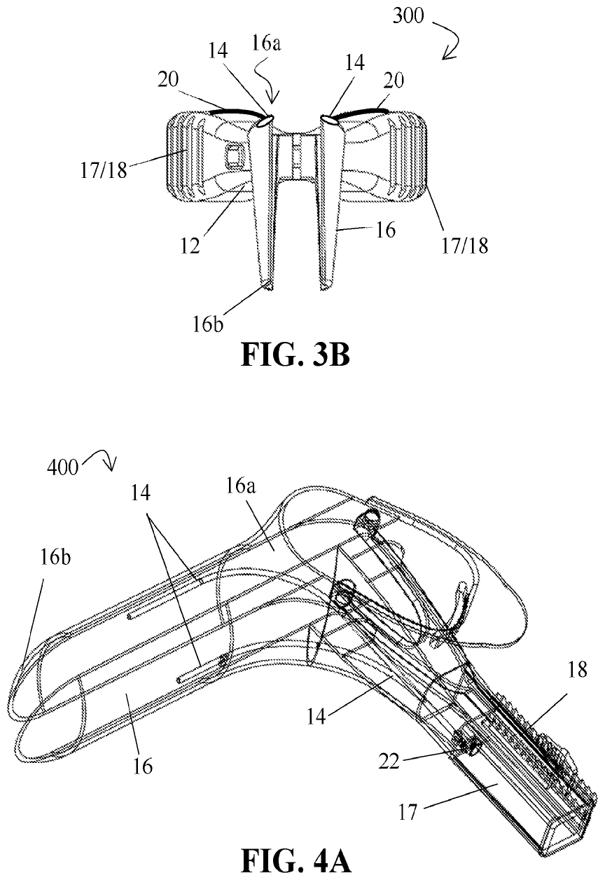

The speculum 12 may be any speculum now known or hereinafter developed in the medical or veterinary arts and, in at least one exemplary embodiment, comprises a nasal speculum as shown in FIGS. 1-3B. The speculum 12 may be formed from any material that is suitable in the medical or veterinary arts, such as metal or plastic. In at least one exemplary embodiment, the speculum 12 comprises a plastic (or other) material as shown in FIGS. 4A and 4B that allows light to pass therethrough to some degree. For example, such material may be clear, transparent, translucent, opaque, or otherwise depending on how much light is desired to pass.

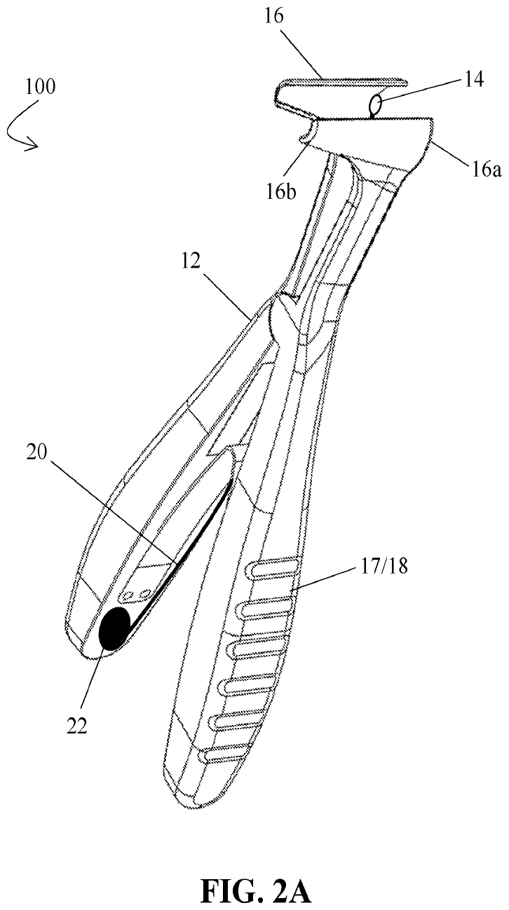

The speculum 12 of FIGS. 1-2B generally comprises a dilation means 16 extending from one or more handle 17. The dilation means 16 comprises a structure extending between a proximal end 16a (proximal to the handle(s) 17) and a distal end 16b, and is configured to move between a closed configuration and an open configuration. For example, the dilation means 16 may comprise two or more blades (as shown with devices 100, 300, 400) or a cylindrical component (not shown) configured to move between a closed configuration having a first diameter and an open configuration having a second, larger diameter.

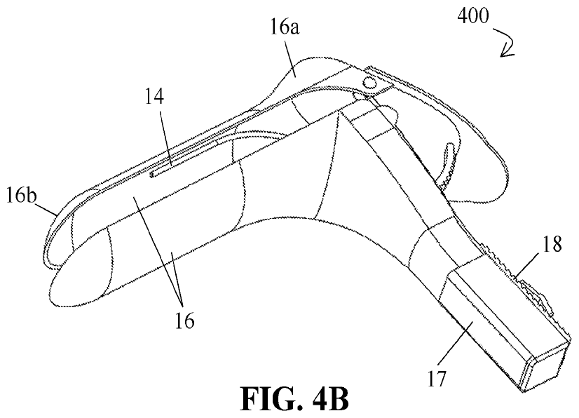

As shown in FIG. 5, in operation, the dilation means 16 is positioned within a body cavity 502 and moved into the open configuration. When positioned in the open configuration, the distal end 16b of dilation means 16 dilates the body cavity 502 and forms a viewing area 504 between the component(s) of the dilation means 16 such that a targeted site beyond the distal end 16b can be easily visualized. In at least one embodiment, the distal end 16b of the dilation means 16 may further comprise a curved configuration (e.g., a duckbill shape) and/or be concave relative to the viewing area 504 (see FIGS. 4A and 4B). This curved configuration increases the surface area of the distal end 16b and is thus more comfortable for the patient as it more evenly distributes pressure within the body cavity 502. Furthermore, the curved configuration effectively holds the surrounding tissue back and creates a larger viewing area 504 when the dilation means 16 is positioned within a body cavity 502 and moved to the open configuration.

Extending from the proximal end 16a of the dilation means 16, the speculum further comprises at least one handle 17 coupled or integrally formed with an actuator 18. The handle 17 is for a practitioner to grip when operating the device 100, 300, 400, and the actuator 18 is configured to move the dilation means 16 between the closed and open configurations and, in at least one embodiment, may further comprise a lock (for locking the dilation means 16 in the desired configuration). The handles 17 and actuator 18 may comprise the same element as shown in FIGS. 1-2B, whereby the handles 17 are biased towards the resting position (i.e. open configuration) shown in FIG. 1. Squeezing the handles 17 together moves the dilation means 16 between its open and closed configurations (FIGS. 2A and 2B illustrating a closed configuration). Alternatively, the handle(s) 17 and actuator 18 may be separate elements as illustrated in the embodiment of FIGS. 4A and 4B (for example, the handle 17 is fixed and the actuator 18 comprises a mechanism for moving the dilation means 16 between the closed and open configurations).

The devices 100, 300, 400 of the present disclosure further comprise at least one light source 14 affixed to the speculum 12 and coupled with a power source 22 via a wire 20. As previously stated, the light source 14 can be used to illuminate a targeted site within the body cavity 502 and, thus, facilitate a practitioner's visualization thereof. The light source 14 may be any light source configured to illuminate when powered by the power source 22. For example, the light source 14 may comprise fluorescent, laser, quartz halogen, LED, xenon, fiber optics, or any other suitable illuminating light source. LED, laser, and fiber optics may be particular suited for these applications due to their stability, light intensity, longevity, and cost-effectiveness.

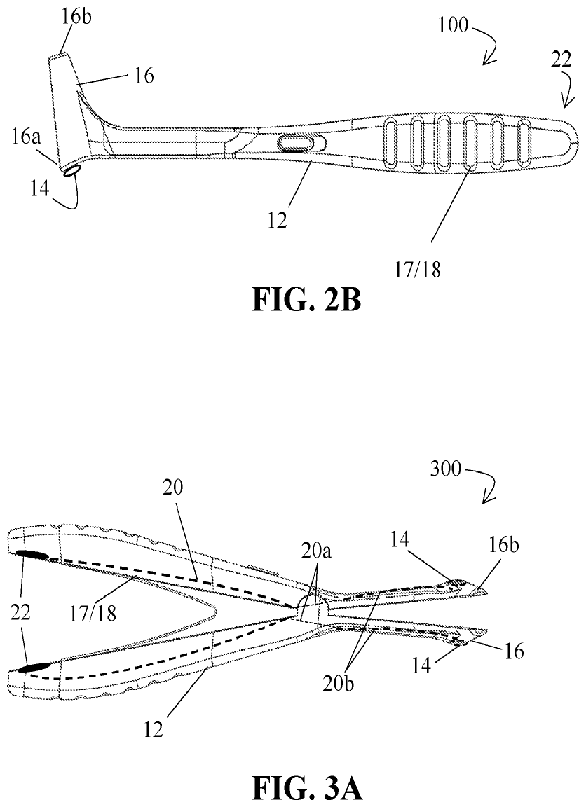

The light source 14 comprises a single bulb or light source that emits direct or diffuse illumination (as shown in FIG. 1), multiple bulbs or light sources (FIGS. 3A and 3B), and/or one or more lighted cables or tubes (FIGS. 4A and 4B) that illuminate along at least part of their lengths and provide more diffuse illumination. The light source 14 may be positioned on or within the speculum 12 at any location where, in operation, the light source 14 can effectively illuminate the targeted site. For example, the light sources 14 shown in FIGS. 1, 3A, and 3B are positioned on the proximal end 16a of the dilation means 16 and emit direct light in the direction of and beyond the distal end 16b. While not illustrated herein, the light source 14 may also be positioned on the distal end 16b of the dilation means 16 or at any position along the length thereof. Additionally or alternatively, the light source 14 may even be positioned within the handle 17 of the device 100, 300, 400, provided that the light source 14 is of sufficient strength (and/or is utilized with one or more mirrors or other reflective or light transferring components) to provide illumination through the interior and/or distal end 16b of the dilation means 16 and, thus, to the targeted site.

In at least the exemplary embodiments shown in FIGS. 4A and 4B, the light source 14 comprises lighted cables or tubes that run along at least part of the length of the dilation means 16 and at least a portion of the speculum 12 comprises a material that allows light to pass therethrough to a desired degree (e.g., a clear or transparent plastic). There, when illuminated, the light source 14 emits diffuse light that is visible through such material of the speculum 12, thus giving the speculum 12 a glowing appearance as the light passes therethrough. Accordingly, in such embodiments, not only the targeted site is illuminated, but the device 400 can illuminate the entire body cavity 502 and viewing area 504. The use of such material for the speculum 12 may be particularly beneficial where the distal end 16b of the dilation means 16 comprises curvature and/or a wider or larger surface area as previously described. In such cases, the increased surface area of the distal end 16b will increase the amount of the dilation means 16 that glows when the light source 14 is illuminated.

The light source 14 may be powered by one or more power sources 22 in electrical communication therewith. In at least one embodiment, each light source 14 is connected to a power source 22 comprising a battery via a wire 20. The wire 20 may be affixed to the side of the handle 17 and/or dilation means 14 as appropriate or, alternatively, the wire 20 may be floating and encased within an interior space defined within the handle 17. It will be noted, however, that a wire 20 may not be necessary in certain configurations; as shown in FIG. 4A, for example, the light source 14 may couple directly with the power source 22 itself. Furthermore, the wire 20 need not comprise a wire configuration at all but may instead comprise any electrically conductive material such as a conductive tape or the like.

Additionally or alternatively, power to the light source 14 may also utilize components suitable for wireless power transfer in addition to—or in lieu of (not shown)—the wire 20 or other direct coupling with the power source 22. Referring back to FIG. 3A, in at least one embodiment, a wire 20 may be utilized in addition to near-field wireless power transfer componentry known in the art (for example, to achieve invective coupling) such that power is transmitted from the power source 22, through the wire 20 (i.e. a primary wire), wirelessly across a gap 20a in wire 20 at or near the hinge of the handle and/or actuator 17/18, and either directly to the light source 14 or through a secondary wire 20b and to the light source 14. Accordingly, the hinge and/or handle 17 may be designed such that the electrical energy from the power source 22 can jump a gap 20a in wire 20 at or near the hinge on the way to the light source 14.

A single power source 22 may be used to power multiple light sources 14 (e.g., a single battery), or each light source 14 may comprise its own power source 22 (see e.g., FIG. 3A). While the power source 22 is shown in the Figures as being positioned on the handle 17 of the speculum 12, power source(s) 22 may be positioned at any location within or on the device 100, 300, 400, as desired, provided that each is in electrical communication (wired or wireless) with a light source 14. Additionally, in at least one exemplary embodiment, the power source 22 is positioned on the device 100, 300, 400 such that it is easily accessible for replacement or other purposes (for example, where the power source 22 comprises a battery). For example, a battery/power source 22 may be removably affixed to an interior wall of the handle 17 with adhesive, Velcro®, a fastener, a battery case, or the like (see device 100 of FIGS. 2A and 2B).

In at least one embodiment, the power source 22 may be any battery capable of storing enough energy to illuminate the light source 14. In at least one exemplary embodiment, the power source 22 comprises a watch battery, an alkaline cell battery, or the like. It will be appreciated that such batteries are inexpensive and, thus, cost effective even if the device 100, 300, 400 is disposable and/or intended for a single use. Instead of a battery, as previously noted, the power source 22 of the device 100, 300, 400 may instead comprise any other suitable power sources now known or hereinafter developed including, without limitation, a corded plug for coupling to a wall outlet or other external power supply.

To promote ease of use, switching the light source 14 on or off should be easily achievable while a practitioner is using the device 100, 300, 400 to dilate a body cavity 502. Accordingly, the device 100, 300, 400 may further comprise a switching mechanism (not shown) for allowing or disrupting electricity flow between the light source 14 and the relevant power source 22. The switching mechanism comprises any switching mechanism known in the art capable of completing and/or interrupting a conducting path between the power source 22 and the light source 14. For example, and without limitation, the switching mechanism can be a toggle (flip switch for continuous “on” or “off”) or momentary (push-for “on” or push-for “off”) type.

The precise configuration of the switching mechanism may vary depending on the design of the light source 14, power source 22, wire 20, and/or other considerations; however, in most cases the switching mechanism is operated manually and positioned on the device 100, 300, 400 such that it can be easily accessed. For example, the switching mechanism may be positioned at or near the handle 17 or proximal end 16a of the dilation means 17. In at least one exemplary embodiment, the switching mechanism is operable to turn the light source 14 on and off using only one finger or touch, thereby facilitating ease of use. Additionally or alternatively, the switching mechanism may be positioned along the outer surface of the dilation means 17 and configured to complete the circuit between the power source 22 and light source 14 upon sensing the application of pressure to the dilation means 17 (e.g., when the device 100, 300, 400 is positioned within a body cavity 502 and moved to the open configuration). In yet another design where the device comprises at least one moveable handle 17, the switching mechanism may be configured to complete the circuit between the power source 22 and light source 14 when the handle(s) 17 are moved together and to interrupt the circuit when the handle(s) 17 return to their resting position. Where the moveable handle(s) 17 are in communication (or integral with) the actuator 18, movement of the handle(s) 17 would not only move the dilation means 16 to the open configuration, but also illuminate the light source 14. Likewise, allowing the handle(s) 17 to return to the resting position moves the dilation means 16 to the closed configuration and extinguishes the light source 14. Due to the unique designs provided herein, a practitioner may deploy the device 100, 300, 400 hereof within a body cavity 502, move the dilation means 16 to the open configuration, and turn on the light source(s) 14 all with a single hand.

In at least one exemplary embodiment, the switching mechanism comprises a plastic strip or button positioned over a power source 22 comprising a battery to disrupt current flow between the battery and the light source 14. There, a practitioner need only remove the plastic strip to complete the circuit (e.g., initiate electrical communication between the battery and the wire 20) and thus illuminate the light source 14. To turn the light source 14 off the plastic strip need only be replaced to again disrupt the current therebetween.

In addition to the embodiments previously described, alternative embodiments of the present disclosure comprise a nasal speculum device comprising a removable light unit (not shown). The removable light unit comprises at least one light source 14, at least one internal power source 22 in electrical communication with the light source(s) 14, any component circuitry or electrical conductance material that may be necessary or appropriate to facilitate electrical connection therebetween, and a switching mechanism to control the illumination of the light source(s) 14. The power source(s) 22, conductance material, at least part of the light source(s) 14, and the switching mechanism are housed within a casing configured to slidably insert into the viewing area 504 of the speculum 12 such that the light source 14 is positioned towards the distal end 16b of the dilation means 16; however, the casing is shaped so as to minimize its obstruction of a practitioner's view through the viewing area 504 when such device is in operation.

In at least one exemplary embodiment of the devices of the present disclosure, the device 100 shown in FIGS. 1, 2A, and 2B comprises a nasal speculum 12 and is formed such that light can traverse through at least a portion of the walls thereof. For example, in at least one exemplary embodiment, portions of the nasal speculum 12 comprise either disposable clear, translucent, or transparent plastic or metal. As shown in FIGS. 1, 3A, and 3B, one or more LED light source(s) 14 may be coupled with the proximal end 16a of the dilation means 16 and directed towards the distal end 16b of the dilation means 16 such that, in operation, the light source(s) 14 can illuminate a nasal cavity and/or a targeted site positioned within such cavity. The device 100 may comprise a single-use device, which prevents the need to sterilize and otherwise clean the device 100 after use. Additionally or alternatively, at least a portion of the walls of the dilation means 16 may comprise one or more perforations such that light can traverse from within the interior of the dilation means 16 to the exterior (and illuminate the surrounding tissue being dilated).

In operation, the devices of the present disclosure may be used to dilate a body cavity as shown in FIG. 5 and illuminate the same, all with a single hand. This provides superior control and ease of use as compared to conventional devices. Furthermore, because the light source 14 and battery/power source 22 may both be internal, there is no cord or separate tool to sterilize, clean, or manage. Among other applications, the devices of the present disclosure are especially suited to treating nosebleeds or epistaxis. The nose has a rich vascular supply, with substantial contributions from the internal carotid artery (ICA) and the external carotid artery (ECA), and is situated in a vulnerable position on the face. As a result, any trauma to the face can cause bleeding, which may be profuse. Posterior nosebleeds, in particular, are caused by bleeding in an artery in the back of the nose. These occur often in the elderly population and may require hospitalization to stop the bleeding.

Nose cauterization is the standard procedure used to treat posterior nosebleeds. Nose cauterization involves identifying the artery that is bleeding and cauterizing the same using an electrocautery instrument or silver nitrate. The devices of the present disclosure are particularly suited to facilitating this treatment in an efficient, clean, cost-effective, and accurate manner.

The devices hereof are also suited for identifying and aiding with the removal of foreign bodies from a body cavity (e.g., toy pieces, candy, rocks, etc. which are often inserted by children) and/or for the evaluation of a targeted site within a body cavity (e.g., evaluation of a nasal tumor). In such cases, the device of the present disclosure may be used to dilate a body cavity and illuminate the same such that a practitioner can visualize a foreign body and/or an anatomical occurrence (such as a tumor) present therein. In such applications, the practitioner can also access and/or remove the foreign body or anatomical growth/occurrence through the viewing area 504 of the speculum 12. A second instrument—for example, forceps, a suction catheter, a biopsy device, and/or a water irrigation device—may be inserted through the viewing area 504 of the speculum 12 to remove the foreign body from the body cavity or remove and/or biopsy an anatomical growth present therein.

Additional instruments may also be employed in conjunction with the devices of the present disclosure. For example, in at least one embodiment, the devices hereof can be used to guide the placement of a nasogastric tube. With conventional devices and techniques, which are inserted blindly, it is common for nasogastric tubes to get stuck against one of the nasal turbinates upon insertion into the nasal cavity. However, using the novel devices of the present disclosure, a practitioner can easily dilate and illuminate the nasal cavity such that, when a nasogastric tube is inserted therein through the viewing area 504, the practitioner can visually navigate the insertion of the tube and guide it around nasal turbinates and other anatomical obstructions. Thereafter, the dilation means 16 may be withdrawn from the body cavity over the tube, thus leaving the tube in place.

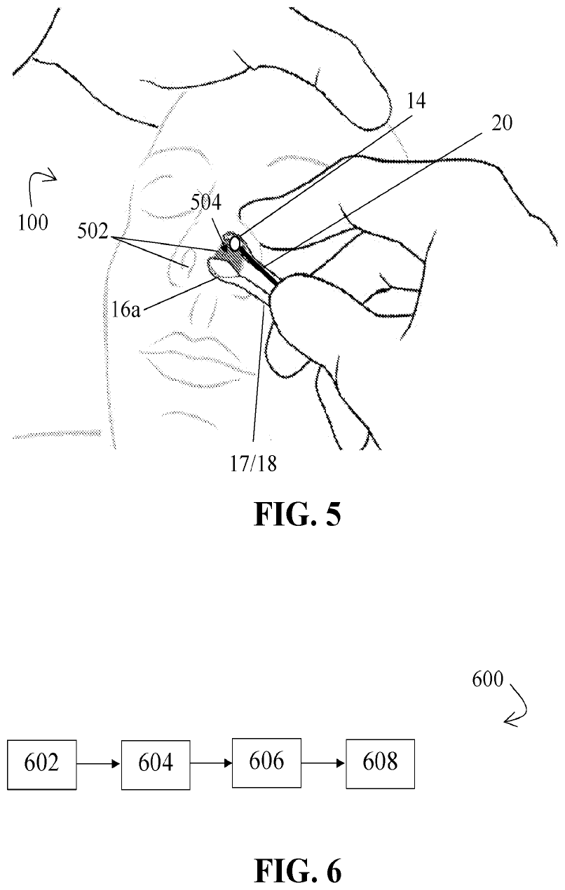

Now referring to FIG. 6, a method 600 for dilating and visualization an orifice using the devices 100, 300, or 400 hereof is also provided. In at least one embodiment, at step 602, a practitioner inserts the dilation means 16 (positioned in a closed configuration) into orifice of the patient to be dilated. When the dilation means 16 is properly positioned, the practitioner operates the handles/actuator 17/18 to move the dilation means 16 from the closed configuration to the open configuration at step 604. Accordingly, at step 604, the orifice is dilated as shown in FIG. 5 and the viewing area 504 is enlarged.

At step 606 the practitioner activates the light source 14 using the switching mechanism, which in at least one exemplary embodiment, can be performed by the same hand the practitioner is using to hold the speculum 12 in place (e.g., the practitioner may be supporting the speculum 12 concurrently with his or her left hand while he or she actuates the light source 14 using his or her left thumb). Activation of the light source 14 illuminates a targeted area distal of the dilation means 16 and/or the orifice itself. For example, where the speculum 12 is formed of a transparent material (such as clear plastic) and/or comprises one or more perforations or holes defined in a wall of the dilation means, the light source 14 may supply direct light towards and through the distal end 16b of the dilation means 16 and diffuse light may travel through the clear blades/components of the dilation means 16 in a radial fashion, thereby causing the speculum 12 itself to glow and illuminating the orifice in locations other than only the viewing area.

Method 600 will now be described in connection with treating a nosebleed using a lighted nasal specula of the present disclosure. While the present embodiment relies upon device 100 in describing the various steps of method 600, any of devices 100, 300, or 400, or embodiments described herein, may be used to perform method 600. Similarly, one of ordinary skill in the art will appreciate that, especially in view of the general example listed above, method 600 may be performed in connection with any body cavity and application is not limited to the nasal cavity specifically described. Furthermore, while the methods hereof are described in connection with the treatment of a human mammal, the devices and methods hereof may be employed in connection with any living being that has an orifice or body cavity suitable for dilation and/or visualization.

At step 602, a practitioner inserts the dilation means 16 (positioned in a closed, or substantially closed, configuration) into the nasal cavity 502 of the patient presenting with the nosebleed to be treated. When the dilation means 16 is properly positioned, the practitioner operates the handles/actuator 17/18 to move the dilation means 16 from the closed configuration to the open configuration at step 604. Accordingly, at step 604, the nasal cavity 502 is dilated as shown in FIG. 5 and the viewing area 504 is enlarged.

At step 606 the practitioner activates the light source 14 using the switching mechanism, which in at least one exemplary embodiment, can be performed by the same hand the practitioner is using to hold the speculum 12 in place (e.g., the practitioner may be supporting the speculum 12 concurrently with his or her left hand while he or she actuates the light source 14 using his or her left thumb). Activation of the light source 14 illuminates a targeted area distal of the dilation means 16 and/or the nasal cavity 502. For example, where the speculum 12 is formed of clear plastic, the light source 14 may supply direct light towards and through the distal end 16b of the dilation means 16 and diffuse light may travel through the clear blades/components of the dilation means 16 in a radial fashion, thereby causing the speculum 12 itself to glow and illuminating the nasal cavity 502 itself.

The light supplied by the light source allows the practitioner to easily identify the bleeding artery (i.e. the targeted site) through the viewing area 504 without the use of a headlamp or other external light source. Once the targeted artery is visualized, at step 608 the practitioner cauterizes the targeted artery using a means known in the art. For example, in at least one embodiment, the practitioner inserts a cauterization stick through the viewing area 504 and past the distal end 16b of the dilation means 16 to cauterize the targeted artery. It will be appreciated that, due to the integral design of the device 100 with the light source 14 and power source 22 coupled directly to the speculum 12, the viewing area 504 remains relatively free of obstruction when in the open configuration, thereby easily allowing for the insertion of one or more tools therethrough.

Exemplary kits are also provided to facilitate commercial use of the present devices and methods. In at least one embodiment, a kit may comprise a lighted specula device 100, 300, 400 packaged within medical packaging comprising a peel seal (plastic-to-plastic or otherwise). There, the lighted specula device 100, 300, 400 may be sterilized prior to placement within the medical packaging such that it is ready for use when opened. The exemplary kits hereof may additionally comprise a cauterization stick such that a practitioner can easily access all of the sterilized tools required for a nosebleed cauterization in one kit. Additionally or alternatively, the kits of the present disclosure may comprise a biopsy tool, a water irrigation device, forceps, a suction catheter, one or more replacement batteries and/or light bulbs for the light source 14, and/or a nasogastric tube.

While various embodiments of the lighted speculum devices, kits, and methods of using the same have been described in considerable detail herein, the embodiments are merely offered as non-limiting examples of the disclosure. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A lighted speculum device comprising:
a dilation component configured to move between a closed configuration and an open configuration;
at least one handle coupled with a proximal end of the dilation component;
a light source coupled with the dilation component, the light source configured to emit light upon activation;
a power source in electrical communication with the light source, the power source coupled directly to one or more of the dilation component, the at least one handle, and the light source; and
a switching mechanism configured to allow or disrupt electricity flow between the light source and the power source.

2. The lighted speculum device of claim 1, wherein the light source is coupled with the proximal end of the dilation component and the power source comprises one or more batteries, each battery coupled directly with an inner side of the at least one handle, and wherein the switching mechanism is operable to activate the light source upon a single touch by a user.

3. The lighted speculum device of claim 1, wherein the switching mechanism is positioned along at least one outer surface of the dilation component and operable to activate the light source upon the application of pressure to the out surface of the dilation component.

4. The lighted speculum device of claim 1, wherein at least a portion of the dilation component comprises a transparent or opaque, disposable plastic material.

5. The lighted speculum device of claim 4, wherein the light source comprises a lighted cable that extends along a length of the dilation component.

6. The lighted speculum device of claim 1, wherein the dilation component comprises at least one wall extending between a proximal end and the distal end of the dilation means, wherein at least a portion of the at least one wall defines a plurality of perforations configured to allow light to traverse therethrough.

7. The lighted speculum device of claim 1, wherein the handle further comprises an actuator configured to cause the dilation component to move between the closed and the open configurations, and the power source is coupled with the light source via wireless power transfer.

8. The lighted speculum device of claim 4, wherein the light source comprises at least one bulb coupled at or near a proximal end of the dilation component.

9. The lighted speculum device of claim 8, wherein the light source is positioned within an interior of the dilation component.

10. A method for dilating and visualizing a nasal orifice, the method comprising:
inserting a dilation means of a lighted speculum device into a nasal orifice of a patient, the dilation means positioned in a closed configuration;
dilating the nasal orifice of the patient and enlarging a viewing area defined by a distal end of the dilation means by moving the dilation means to an open configuration;
activating a light source of the lighted speculum to emit light at least through the distal end of the dilation means and into the nasal orifice; and
visualizing a targeted site within the nasal orifice through the viewing area;
wherein a power source of the light source is positioned within or on the lighted speculum.

11. The method of claim 10, wherein:
the dilation means comprises at least one wall extending between a proximal end of the dilation means and the distal end of the dilation means, at least a portion of the at least one wall comprises material that allows light to pass therethrough; and
the step of activating the light source further comprises illuminating a portion of the dilated nasal orifice other than the viewing area by emitting diffuse light through the portion of the at least one wall.

12. The method of claim 10, wherein the light source is affixed to the dilation means.

13. The method of claim 11, wherein the light source comprises a lighted cable or wire that extends along a length of the dilation means.

14. The method of claim 10, wherein the step of activating a light source of the lighted speculum comprises operating a switching mechanism configured to allow or disrupt electrical flow between the light source and the power source.

15. The method of claim 11, wherein the nasal orifice is a nasal cavity, the targeted site is a bleeding artery, and the method further comprises the step of inserting a cauterization tool through the viewing area and cauterizing the bleeding artery.

16. A dilation kit comprising a lighted nasal speculum comprising:
a dilation component comprising at least two blades, each blade comprising:
a proximal end,
a distal end, and
at least one wall,
wherein the distal ends of the blades define a viewing area at least when the dilation component is in an open configuration, and at least one the walls of the blades comprises at least a portion that allows light to pass therethrough and defines an interior space that extends between the proximal ends and the distal ends of the blades; and
a light source coupled with the dilation component such that light emitted therefrom illuminates the viewing area and passes through the portion of the at least one wall of the dilation component;
wherein the blades of the dilation component are configured to move laterally between a closed configuration and the open configuration.

17. The dilation kit of claim 16, further comprising a cauterization stick, a distal end of the cauterization stick configured to be insertable within the interior space and through the viewing area of the dilation component of the lighted speculum.

18. The dilation kit of claim 17, further comprising a nasogastric tube.

19. The dilation kit of claim 16, further comprising one or more of a biopsy tool, a water irrigation device, forceps, and a suction catheter.

20. The dilation kit of claim 16, wherein:
the lighted speculum further comprises at least one battery in electrical communication with the light source,
the light source comprises at least one bulb,
the blades of the dilation component comprise (a) one or more perforations therein, (b) a transparent, translucent, or opaque material to allow light to traverse therethrough, or (c) both (a) and (b), and
the kit further comprises one or more replacement batteries, one or more replacement bulbs, or both.
